(19) United States
(12) Patent Application Publication
(10) Pub. No.: US 2012/0328530 A1
(43) Pub. Date: Dec. 27, 2012

(54) TOLUIDINE BLUE DERIVATIVES AS PHOTOSENSITISING COMPOUNDS

(75) Inventor: Mark Wainwright, Lancashire (GB)

(73) Assignee: PHARMALUCIA LIMITED, Merseyside (GB)

(21) Appl. No.: 13/583,299

(22) PCT Filed: Mar. 11, 2011
(86) PCT No.: PCT/GB2011/050481

§ 371 Date: Sep. 7, 2012

(30) Foreign Application Priority Data
Mar. 17, 2010 (GB) 1004450.1

(51) Int. Cl. C07D 279/20; C07D 498/04; C07D 517/04; A61K 31/5415; A61K 49/00; G01N 21/64; A61P 35/00; A61P 31/00; A61P 31/12; A61P 31/04; A61P 31/10; A61P 33/02; A61L 2/08; A61M 37/00; C07D 513/04

(52) U.S. Cl. 424/9.6; 544/37; 544/31; 544/99; 544/1; 540/578; 514/224.8; 435/34; 435/5; 422/22; 604/20

(57) ABSTRACT

A compound of formula (III), or a pharmaceutically acceptable derivative thereof for use in a method of combating and/or detecting a pathogen and/or tumour cells; wherein X is selected from O, S and Se; each of R², R³ and R⁴ is independently selected from hydrogen or an optionally substituted alkyl, alkenyl, alkynyl or aryl group; and R¹ is selected from halogen, sulfo, acyl, sulfoxy, mercapto, nitro, amino, hydroxy or an optionally substituted alkyl, alkenyl, alkynyl, aryl, amine or alkoxy group; wherein R¹ is not methyl or hydrogen when each of R³ and R⁴ is methyl or hydrogen.

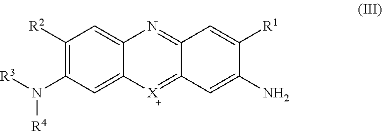

The present invention relates to novel compounds, compositions comprising these and other compounds, and to methods and uses relating thereto. In particular the present invention relates to photosensitising compounds, especially those which induce the formation of reactive oxygen species on exposure to light of a particular wavelength. The compounds of the present invention have been found to be useful in methods for combating pathogens, especially bacterial, and in methods for detecting pathogens.

Methylene blue has the structure shown in formula (I) and has been known as a photosensitising antiviral agent since the 1930s.

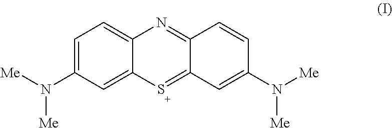

However the use of methylene blue as a virucidal agent has some disadvantages, for example in terms of collateral effects to host cells and thus there is a continuing need to provide improved antiviral agents.

Toluidine blue has the structure shown in formula (II)

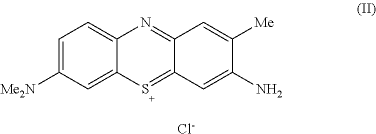

This compound is a known photosentising antibacterial agent. However it is activated using light having a wavelength of 630 nm. Unfortunately light of this wavelength is readily absorbed by haemoglobin in the blood and thus it may be difficult to activate toluidine blue in the body or in samples containing blood. It would therefore be useful to provide compounds having antipathogenic activity which may be photoactivated at wavelengths of greater than 630 nm.

The present invention seeks to provide compounds and compositions having improved properties.

According to a first aspect of the present invention there is provided a compound of formula (III):

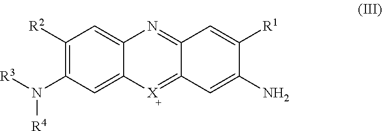

or a pharmaceutically acceptable derivative thereof for use in a method of combating and/or detecting a pathogen and/or tumour cells;
wherein X is selected from O, S and Se; each of R², R³ and R⁴ is independently selected from hydrogen or an optionally substituted alkyl, alkenyl, alkynyl or aryl group; and R¹ is selected from halogen, sulfo, acyl, sulfoxy, mercapto, nitro, amino, hydroxy or an optionally substituted alkyl, alkenyl, alkynyl, aryl, amine or alkoxy group; wherein R¹ is not methyl or hydrogen when each of R³ and R⁴ is methyl or hydrogen.

For the avoidance of doubt the compounds of formula (III) do not include compounds in which R³═R⁴=H and R¹ is Me or H; R³═R⁴=Me and R¹ is Me or H; or R³=Me, R⁴=H and R¹ is Me or H.

Pharmaceutically acceptable derivatives include salts and solvates, such as acid addition salts. Thus the compounds of the invention may include an anion, monovalent or polyvalent, sufficient to balance the charge on the compound of formula (III). Examples of suitable counter ions include both organic and inorganic moieties. Suitable organic moieties include acetate, citrate and tartrate. Suitable inorganic moieties include halide, carboxylate, sulphate and phosphate counterions. Especially preferred counterions are sulphate and chloride.

In some preferred embodiments X is sulphur or selenium, most preferably sulphur.

In some preferred embodiments, X is oxygen.

When any of R², R³, or R⁴ is an alkyl group, it is preferably an alkyl group having from 1 to 30 carbon atoms, preferably 1 to 20 carbon atoms, more preferably 1 to 12, for example 1 to 8 carbon atoms, preferably 1 to 6 carbon atoms and most preferably from 1 to 4 carbon atoms.

When R¹ is an alkyl group it is preferably an alkyl group having from 2 to 30 carbon atoms, preferably 2 to 20 carbon atoms, more preferably 2 to 12 carbon atoms, for example from 2 to 8 carbon atoms, preferably 2 to 6 carbon atoms and most preferably 2 to 4 carbon atoms.

The or each such alkyl group may be straight chained or branched and may be optionally substituted with one or more substituents selected from halogen, nitro, sulfoxy, sulfo, amino, acyl, hydroxy, alkoxy (especially C₁ to C₄ alkoxy), mercapto or a silicon containing group.

When any of R¹, R², R³ or R⁴ is an alkenyl group, it is preferably an alkenyl group having from 2 to 30 carbon atoms, preferably 2 to 20 carbon atoms, more preferably 2 to 12, for example 2 to 8, preferably 2 to 6 carbon atoms and most preferably from 2 to 4 carbon atoms.

The or each such alkenyl group may be straight chained or branched and may have an E or a Z configuration. Each such alkenyl group may be optionally substituted with one more substituents selected from halogen, nitro, sulfo, sulfoxy, amino, acyl, hydroxy, alkoxy (especially C₁ to C₄ alkoxy), mercapto or a silicon containing group.

When any of R¹, R², R³ or R⁴ is an alkynyl group, it is preferably an alkynyl group having from 2 to 30 carbon atoms, preferably 2 to 20 carbon atoms, more preferably 2 to 12, for example 2 to 8 carbon atoms, preferably 2 to 6 carbon atoms and most preferably 2 to 4 carbon atoms.

The or each such alkynyl group may be straight chained or branched and may be optionally substituted with one or more substituents selected from halogen, nitro, sulfo, sulfoxy, amino, acyl, hydroxy, alkoxy (especially C1 to C4 alkoxy), mercapto or a silicon containing group.

When any of R¹, R², R³ or R⁴ is an aryl group, it may be an aryl group comprising only carbon atoms within the aromatic ring or it may be a heteroaromatic moiety including one or more heteroatoms selected from nitrogen, sulphur and oxygen. Preferably the or each aryl group has between 3 and 15 atoms in the aromatic ring, preferably between 5 and 10 atoms. The or each such aryl group may be optionally substituted with one or more substituents selected from halogen, nitro, sulfo, sulfoxy, amino, hydroxyl, acyl, alkoxy (especially C1 to C4 alkoxy), alkyl (especially C1 to C5 alkyl), mercapto or a silicon containing group.

Preferably when any of R¹, R², R³ or R⁴ is an aryl group it is an aryl group comprising only carbon atoms in the aromatic ring. Preferably it comprises from 6 to 10 carbon atoms.

In some preferred embodiments R¹, R², R³ or R⁴ may be an alkylaryl group for example an optionally substituted benzyl group. Such compounds are within the definition of aryl groups of the present invention.

When any of R¹ is an alkoxy group, it is preferably an alkoxy group having from 1 to 30 carbon atoms, preferably 1 to 20 carbon atoms, more preferably 1 to 12, for example 1 to 8 carbon atoms, preferably 1 to 6 carbon atoms and most preferably 1 to 4 carbon atoms. Each alkyl group of an alkoxy substituent may be straight chained or branched and may be substituted with one or more substituents selected from halogen, nitro, amino, acyl, hydroxy, alkoxy (especially C1 to C4 alkoxy), mercapto or a silicon containing group.

Suitable silicon containing groups are those of formula SiA₃ in which each A is independently selected from an optionally substituted alkyl, alkenyl, alkynyl or alkoxy group. Any such optionally substituted alkyl, alkenyl, alkynyl or alkoxy group may be as defined above. Preferably each A is independently selected from optionally substituted alkyl or alkoxy groups. More preferably each is independently selected from unsubstituted alkyl or alkoxy groups, preferably those having 1 to 10 carbon atoms, more preferably 1 to 6 carbon atoms, for examples 1 to 4 carbon atoms.

Optionally substituted alkyl, alkenyl, alkynyl or alkoxy groups may be substituted along the length of the chain and/or at the end of the chain to include a terminal substituent. Alternatively and/or additionally each or any of these groups may be substituted within the chain with one or more hetero atoms, for example oxygen, nitrogen or sulphur to form an ether, thioether or amino linkage, or silicon. In embodiments in which the alkyl, alkenyl, alkynyl or alkoxy group includes a silicon atom in the chain, this may be directed bonded to two carbon atoms of the chain or it may be bonded via an oxygen linkage to form a silyl ether within the chain. In addition to the groups forming part of the alkyl, alkenyl, alkynyl or alkoxy chain, the silicon atom will be bonded to two further groups, or three further groups in the case of a terminal silicon substituent. These further groups may comprise any suitable group as would be known to the person skilled in the art. However in preferred embodiments the further groups are independently selected from optionally substituted alkyl or alkoxy groups. More preferably they are independently selected from unsubstituted alkyl or alkoxy groups, preferably those having 1 to 10 carbon atoms, more preferably 1 to 6 carbon atoms, for example 1 to 4 carbon atoms.

When R¹ is an optionally substituted amine group, it may be an alkyl amine, alkenyl amine, alkynyl amine, aryl amine or alkoxy amine moiety. Any of R¹, R², R³ or R⁴, may by a group of formula R⁵NR⁶R⁷ wherein R⁵ is selected from a bond, or an optionally substituted alkylene, alkenylene, alkynylene or arylene moiety and each of R⁶ and R⁷ is independently selected from hydrogen or an optionally substituted alkyl, alkenyl, alkynyl, aryl or alkoxy residue. Preferably R⁵ is selected from an optionally substituted alkylene or alkenylene group, most preferably an optionally substituted alkylene group, especially an alkylene group having 1 to 12 carbon atoms, preferably 1 to 10, for example 1 to 8, more preferably 1 to 6 and most preferably 1 to 4 carbon atoms, for example 2 or 3 carbon atoms. Such an alkylene group be straight chained or branched. It may be substituted with one or more substituents selected from halogen, nitro, amino, acyl, hydroxy, alkoxy (especially C1 to C4 alkoxy), mercapto or a silicon containing group (as previously defined herein). Preferably it is unsubstituted. Preferably it is not branched.

Each of R⁶ and R⁷ may be independently selected from hydrogen, and an optionally substituted alkyl, alkenyl, alkynyl, aryl or alkoxy group. Preferably each is independently selected from an optionally substituted alkyl or alkenyl group, preferably having up to 30 carbon atoms, preferably up to 24 carbon atoms, for example 18 carbon atoms, more preferably up to 12 carbon atoms, preferably up to 8 carbon atoms, more preferably up to 6 carbon atoms. Such optionally substituted alkyl or alkenyl groups may be straight chained or branched. Preferably they are straight chained. When either or each of R⁶ or R⁷ is an alkyl group it is preferably an alkyl group having 1 to 4 carbon atoms, for example methyl, ethyl, propyl (especially isopropyl) or butyl (especially tertiary butyl). In embodiments in which R⁶ and/or R⁷ is alkenyl, each is preferably an alkenyl group having 2 to 4 carbon atoms. In embodiments in which R⁶ and/or R⁷ is an alkenyl group, each double bond may have an E or a Z configuration.

In some embodiments R³ and R⁴ may be linked to form a ring. They may form an aromatic ring or an aliphatic ring. Suitably in such embodiments R³ and R⁴ are linked to form a ring including from 3 to 8 atoms (the total number of atoms in the ring). The ring may include one or more further hetero atoms within the ring for example O, S, N or Si (in addition to nitrogen of the group NR³R⁴). The ring may be optionally substituted for example with one or more substituents selected from halogen, nitro, sulfo, sulfoxy, amino, acyl, hydroxy, mercapto, alkoxy (especially C1 to C4 alkoxy) mercapto or a silicon containing group (as previously defined herein). In some preferred embodiments R³ and R⁴ are joined to form a 6-membered ring.

In some embodiments R³ or R⁴ and R² may be linked to form a ring. They may form an aromatic ring or an aliphatic ring. Suitably in such embodiments R³ and R² are linked to form a ring including from 5 to 7 atoms (the total number of atoms in the ring). The ring may include one or more further hetero atoms within the ring for example O, S, N or Si (in addition to the nitrogen atom of the group NR³R⁴), preferably including 6 atoms. The ring may be optionally substituted for example with one or more substituents selected from halogen, nitro, sulfo, sulfoxy, amino, acyl, hydroxy, mercapto, alkyl (especially C1 to C4 alkyl), alkoxy (especially C1 to C4 alkoxy), or a silicon containing group (as previously defined herein). In some preferred embodiments the ring is an optionally substituted aliphatic ring which comprises no further hetero atom in the ring.

In some preferred embodiments R¹ is selected from an optionally substituted alkyl group having at least 2 carbon atoms, an optionally substituted aryl group and an optionally substituted alkoxy group. The alkyl group of the alkyl or alkoxy group may be straight-chained or branched and may be substituted with one or more substituents selected from halogen, nitro, sulfo, sulfoxy, hydroxyl, acyl, alkoxy (especially C₁ to C₄ alkoxy), amino, mercapto or a silicon containing group (as previously defined herein).

When R¹ is alkyl it is preferably an alkyl group having from 2 to 12 carbon atoms, preferably 2 to 8 carbon atoms, suitably from 2 to 5 carbon atoms. Suitably R¹ may be selected from ethyl, n-propyl, isopropyl, n-butyl, sec-butyl, t-butyl and isobutyl. R¹ may preferably be selected from ethyl, n-propyl and t-butyl.

When R¹ is alkoxy it is suitably a C₁ to C₁₀ alkoxy group, preferably a C₁ to C₆ alkoxy group. Suitably it is selected from methoxy, ethoxy, propoxy (especially n-propoxy) and butoxy (especially t-butoxy).

When R¹ is an aryl group it is preferably a phenyl containing aryl group. It may be phenyl or an alkylaryl group of formula (CH₂)ₙAr in which n may be from 1 to 10, preferably 1 to 6, most preferably 1 to 4 and Ar is optionally substituted phenyl or napthyl. Especially preferred aryl groups are phenyl and benzyl.

In especially preferred embodiments R¹ is selected from ethyl, propyl (especially n-propyl), butyl (especially t-butyl), phenyl, benzyl, methoxy and ethoxy.

Preferably each of R², R³ and R⁴ is independently selected from hydrogen, and an optionally substituted alkyl group.

Preferably when any of R², R³ or R⁴ is an alkyl group it is an alkyl group having from 1 to 10, preferably 1 to 6, most preferably 1 to 4 carbon atoms. Thus each of R², R³ and R⁴ may be independently selected from methyl, ethyl, propyl (especially isopropyl), and butyl (especially tertiary butyl).

When any of R², R³ or R⁴ is alkyl, the alkyl group may be straight-chained or branched and may be optionally substituted with one or more groups selected from halogen, amino, hydroxyl, acyl, sulfo, sulfoxy, mercapto, alkoxy (especially C1 to C4 alkoxy) or a silicon containing group (as previously defined herein). A substituent may be along the alkyl chain of the alkyl group or at the end of the chain. In some preferred embodiments, a terminal hydroxy or amino group is present. Thus each of R², R³ or R⁴ may be independently selected from a group of formula —(CH₂)ₙ₊ₘOH or —(CH₂)ₙO(CH₂)ₘOH or —(CH₂)ₙO(CH₂)ₘCH₃. where n+m is from 2 to 10, preferably 2 to 6, for example 2 to 4.

In some preferred embodiments R² is hydrogen.

If R¹ is methyl or hydrogen, at least one of R³ and R⁴ is not hydrogen. Preferably R³ is not hydrogen and R⁴ is not hydrogen.

In some preferred embodiments R³ is the same as R⁴.

In especially preferred embodiments R³ is C₁ to C₄ alkyl. R³ is suitably selected from methyl, ethyl, n-propyl, n-butyl, t-butyl, n-pentyl and n-hexyl. Most preferably R³ is methyl or ethyl.

In especially preferred embodiments R⁴ is C₁ to C₄ alkyl. R⁴ is suitably selected from methyl, ethyl, n-propyl, n-butyl, t-butyl, n-pentyl and n-hexyl. Most preferably R⁴ is methyl or ethyl.

In some preferred embodiments R² and R³ are joined to provide an alkylene chain and thus form an aliphatic ring; and R⁴ is hydrogen or C₁ to C₆ alkyl, preferably C₁ to C₄ alkyl.

Thus the compound of formula (III) may be represented by formula (IV):

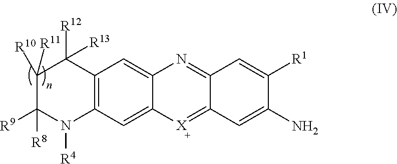

wherein n is suitably 0 to 2, preferably 1, R⁴ is preferably alkyl, R¹ is as previously defined herein and each of R⁸, R⁹, R¹⁰R¹¹, R¹² and R¹³ is independently hydrogen or an optionally substituted alkyl, alkenyl, alkoxy or aryl group. Preferably each of R⁸, R⁹, R¹⁰, R¹¹, R¹² and R¹³ is independently hydrogen or a C₁ to C₄ alkyl group. Most preferably each R¹⁰ and each R¹¹ is hydrogen. Preferably R⁸ is a C₁ to C₄ alkyl group, suitably methyl. Preferably R⁹ is a C₁ to C₄ alkyl group, suitably methyl. Preferably R¹² is hydrogen and R¹³ is a C₁ to C₄ alkyl group, suitably methyl.

The first aspect of the present invention provides compounds for use in a method of combating and/or detecting a pathogen and/or tumour cells.

The pathogen may be selected from viruses, bacteria, fungi and protozoa.

In some preferred embodiments the first aspect of the present invention provides compounds of formula (III) for use in a method of combating and/or detecting bacteria.

Suitably the present invention provides compounds for use in a method of combating and/or detecting bacteria selected from Gram positive bacteria, Gram negative bacteria and mycobacteria.

The compounds of the present invention may be used in a method of combating and/or detecting a bacteria selected from, but not limited to, Staphylococous aureus, Enterococous faecalis, Escherichia coli, Proteus mirabilis, Clostridium difficile, Acinetobacter baumannii, Pseudomonas aeruginosa, Propionibacterium acnes, Staphylococcus epidermidis and Corynebacterium acnes.

By a method of combating bacteria we mean to include methods which kill bacteria and/or methods which inhibit the growth of bacteria and/or methods which prevent the growth of bacteria.

The compounds of formula (III) may be regarded as antibacterial agents. Thus the present invention further provides compounds of formula (III) for use as an antibacterial agent.

In some preferred embodiments the first aspect of the present invention provides compounds of formula (III) for use in a method of combating and/or detecting fungi.

Suitably the present invention provides compounds for use in a method of combating and/or detecting a fungal species selected from, but not limited to Candida, Aspergillus, Cryptococcus, Histoplasma, Pneumocystis, Stachybotrys and Pityrosporum spp.

By a method of combating fungi we mean to include methods which kill fungi and/or methods which inhibit the growth of fungi and/or methods which prevent the growth of fungi.

The compounds of formula (III) may be regarded as antifungal agents. Thus the present invention further provides compounds of formula (III) for use as an antifungal agent.

The present invention may provide a method of combating and/or detecting protozoal species which can cause infection.

By a method of combating protozoal species we mean to include methods which kill protozoal species and/or methods which inhibit the growth or protozoal species and/or methods which prevent the growth of protozoal species.

Suitably the present invention provides compounds for use in combating and/or detecting a protozoal species selected from Plasmodium, Leishmania and Trypanosoma spp.

In an especially preferred embodiment, the first aspect of the present invention provides compounds of formula (III) for use in a method of combating and/or detecting a virus.

Suitably the present invention provides compounds for use in a method of combating and/or detecting a virus selected human immunodeficiency viruses, common cold viruses, influenza, herpes, hepatitis and West Nile viruses.

By a method of combating a virus we mean to include methods which kill a virus and/or methods which inhibit the growth of a virus and/or methods which prevent the growth of a virus.

The compounds of formula (III) may be regarded as antiviral agents. Thus the present invention further provides compounds of formula (III) for use as an antiviral agent.

Compounds effective for combating a virus and/or bacteria and/or a fungus include those compounds of formula (III) in which X is S or Se.

The compounds used in the present invention are photosensitising and it is believed that the anti-pathogenic activity is achieved upon exposure to light. Exposing the compounds to light of a specific wavelength suitably causes the formation of reactive oxygen species, for example singlet oxygen, hydroxyl radicals or superoxide.

By photosensitising we mean to refer to compounds which are reactive to light, especially light of a particular wavelength, but do not necessarily decompose upon exposure to light.

Thus the present invention provides compounds of formula (III) for use in a method of combating and/or detecting a pathogen wherein the method comprises exposing the compounds to light of an appropriate wavelength in the presence of oxygen.

It is believed that the singlet oxygen produced upon irradiation with light is involved in combating a pathogen.

In some embodiments irradiation with light does not lead to the formation of singlet oxygen but does cause the compound to fluoresce. In such embodiments the compounds may be used in a method of detecting a pathogen. Preferred compounds for use in detecting a pathogen are those of formula (III) in which X is O. Especially useful in detection methods are compounds in which X is O.

Suitably the compounds of formula (III) are activated upon exposure to light having a wavelength of from 500 to 900 nm, preferably 550 to 850 nm, for example 600 to 750 nm, more preferably 620 to 700 nm. Light of the desired wavelength may suitably be provided by an LED or a laser. It is most preferred that the compounds of formula (III) are activated upon exposure to light having a wavelength of at least 630 nm, preferably at least 632 nm, more preferably at least 634 nm.

Without wishing to be bound by theory it is believed that the phenothiazinium moiety of the compounds of formula (III) is able to intercalate into the nucleic acid of viruses. Irradiation with light produces singlet oxygen which then damages the DNA. It is believed that the inclusion of basic side chains helps anchor the compounds of formula (III) to the nucleic acid backbone in viruses. The killing process in other microbial types may include action against nucleic acid, cell wall, ribosome, mitochondria or other organelles.

In some embodiments the compounds of the present invention may be used in a method of combating and/or detecting a pathogen for example a virus, fungus or bacterium, in a blood product. Suitably the blood product is contacted with the compound of formula (III) and then irradiated with light of an appropriate wavelength, for example 600 to 800 nm, preferably greater than 630 nm. Thus the present invention suitably provides a method of reducing the level of a pathogenic contaminant in a blood product.

By a blood product we mean to refer to whole blood or a component of whole blood, for example cellular blood components (including red blood cells and platelets), blood proteins (for example blood clotting factors, enzymes, albumin, plasminogen, and immunoglobins) and liquid blood components (for example plasma and plasma-containing compositions).

The present invention may provide a method of combating pathogens in a blood product. It may also provide a method of detecting pathogenic species for example, a virus, bacterium or fungus in a blood product. In such a detection method irradiation with light of a particular wavelength enables the compound of formula (III) to be seen but it does not produce singlet oxygen.

In some embodiments the compounds of formula (III) are included in a composition suitable for administration to a patient infected with a pathogen.

The present invention also provides compounds of formula (III) for use in a method of combating and/or detecting tumour cells.

The present invention is particularly useful in methods of combating and/or detecting tumour at or near to a surface of the body.

The methods of the present invention may also include the detection of pre-cancerous cells, that is cells likely to develop into tumour cells. References in this specification to methods of detecting tumour cells include methods of detecting pre-cancerous cells.

The compounds of the present invention may be useful in methods of combating and/or detecting tumour cells at or near to an internal or external surface of the body. For example it may be useful in combating and/or detecting tumour cells (including pre-cancerous cells) in the skin, in the mouth, in the bladder, in the vagina, in the anus, in the throat, in the oesophagus, in the bowel or in the cervix.

Where the compounds are used in a method of combating tumour cells, a composition comprising a compound of formula (III) may suitably be applied to a bodily surface at which tumour cells are known to be present. The compound is then suitably activated by application of light of an appropriate wavelength (suitably 600 to 800 nm, preferably greater than 630 nm or greater than 634 nm). Activation of the photosensitising compounds suitably causes the formation of reactive oxygen species. This reactive oxygen species is believed to kill tumour cells by necrosis and/or apoptosis. Without wishing to be bound by theory it is believed that the compounds of the present invention have an affinity for tumour cells and thus form an interaction with these cells. The formation of active oxygen is therefore specifically targeted at the tumour cells.

Compounds of formula (III) which are especially useful in methods of combating tumour cells include those in which X is S or Se. By methods of combating tumour cells we mean to refer to the killing of tumour cells, the prevention of growth of existing tumour cells and preventing the formation of new tumour cells.

Compounds of formula (III) in which X is O are particularly useful in methods of detecting tumour cells (including precancerous cells). Such compounds suitably fluoresce upon application of light of an appropriate wavelength (preferably greater than 630 nm). Thus the detecting method suitably involves applying a composition comprising a compound of formula (III) to bodily surface at which it is suspected there may be present tumour cells (or precancerous cells). Without wishing to be bound by theory it is believed that the compounds of formula (III) have an affinity for tumour cells and/or pre-cancerous cells. Subsequent application of light of an appropriate wavelength causes fluorescence of the compound of formula (III) thus revealing the location of tumour cells (or precancerous cells).

For example the compounds of the present invention may be used in a method of detecting tumour cells in the mouth. If a patient rinses their mouth with a composition comprising a compound of formula (III), the compound will interact with any tumour cells (or pre-cancerous cells) present. Applying light of the appropriate wavelength to the mouth will then cause the compound of formula (III) to fluoresce revealing the presence or otherwise of any tumour cells.

According to a second aspect of the present invention there is provided a composition comprising a compound of formula (III) as defined in relation to the first aspect and a pharmaceutically acceptable carrier.

The composition of the second aspect may be provided in any suitable form depending upon the desired application. Preferably the composition is provided in a form suitable for topical application. For example it may be provided in the form of a gel, paste, lotion, powder, solution, cream or the like.

The composition of the present invention may comprise any suitable pharmaceutically acceptable carrier.

The pharmaceutically acceptable carrier may be aqueous based or it may be based on an organic solvent. It may comprise water, an organic solvent or a mixture thereof. In preferred embodiments the compound of formula (III) is completely dissolved in the carrier.

Preferred organic solvents for inclusion in the carrier are water miscible solvents, for example an alcohol such as a C₁ to C₄ alcohol (methanol, ethanol, n-proponol, isoproponol, n-butanol and sec-butanol); amides; ketones (for example acetone and methyl and ether ketone); water miscible ethers; and diols (especially diols having 1 to 12 carbon atoms, for example ethylene glycol).

Suitably the composition comprises from 0.0001 to 50 wt % of one or more compounds of formula (III), preferably from 0.005 to 20 wt %, preferably from 0.01 to 15 wt %, more preferably from 0.005 to 10 wt %, for example from 0.1 to 5 wt %.

When a composition of the present invention is used topically to treat a viral infection, the composition is suitably applied to the infected area and then light of the appropriate wavelength is applied. This will typically be carried out by a skilled healthcare professional who is appropriately trained. The composition may be used in a similar manner to topically treat a bacterial or fungal infection or an infection caused by a protozoal species.

Although the compounds of the present invention are preferably provided in a composition suitable for external topical use, alternative methods in which the compounds can be delivered to the targeted sites and irradiated are also within the scope of the invention.

In an especially preferred embodiment the compositions of the second aspect of the present invention are used in a method of diagnosis.

For example the composition may comprise a solution for rinsing the mouth for use in a method to reveal any tumour cells (including pre-cancerous cells). Such a mouth-wash composition may suitably comprise saline solution and a compound of formula (III).

According to a third aspect of the present invention, there is provided compounds of formula (III).

Preferred features of the second and third aspects of the present invention are as defined in relation to the first aspect.

According to a fourth aspect of the present invention there is provided a method of preparing a compound of formula (III).

In embodiments in which X is S, the compound of formula (III) is preferably prepared by reacting a compound of formula (VI) with a compound of formula (VII), suitably under oxidative conditions:

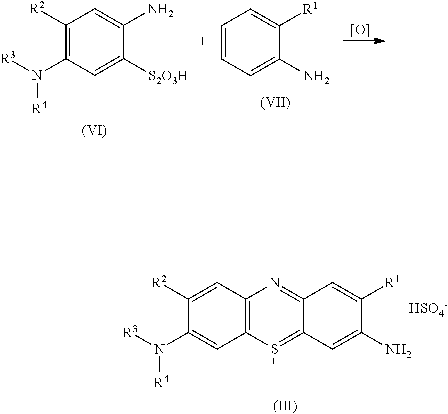

The reaction suitably involves heating a solution comprising a compound of formula (VI) and a compound of formula (VII) in the presence of a suitable oxidising agent. Suitable solvents include protic solvents, for example alcohols. One suitable solvent is methanol. The reaction mixture may be heated at a temperature of from 30 to 100° C., for example from 40 to 80° C., suitably from 55 to 70° C. The reaction mixture may be heated for a period of from 0.1 to 12 h, preferably from 0.25 to 6 h, suitably from 0.5 to 2 h. Preferably a slight molar excess of the compound of formula (VII) is used (suitably 1.05 to 1.5 molar equivalents compared to the compound of formula (VI), for example 1.2 to 1.3 molar equivalents). Any suitable oxidising agent may be used. A particularly preferred oxidising agent is silver carbonate on celite.

In embodiments in which X is O the compound of formula (III) is preferably prepared by reacting a compound of formula (VIII) with a compound of formula (IX), suitably under oxidative conditions:

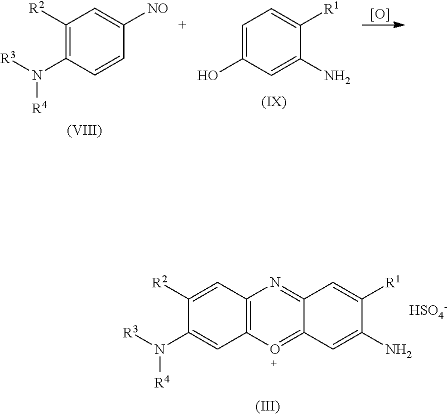

Similar reaction conditions to those described in relation to the reaction of the compound of formula (VI) and the compound of formula (VII) could be used.

In embodiments in which X is Se the compound of formula (III) is preferably prepared by reacting a compound of formula (X) with a compound of formula (XI), suitably under oxidative conditions:

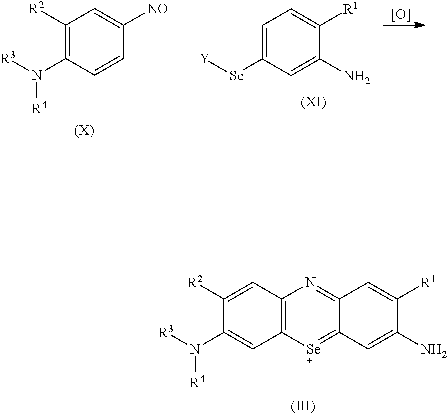

Similar reaction conditions to those described in relation to the reaction of the compound of formula (VI) and the compound of formula (VII) could be used.

The above reaction conditions are merely illustrative and the person skilled in the art would be able to vary these as appropriate.

According to a fifth aspect of the present invention there is provided a method of treating or diagnosing an animal having a disease caused by a pathogen or tumour cells, the method comprising administering to the animal a compound of formula (III). Preferably the method of the fifth aspect comprises administering to the animal a composition of the second aspect. Preferably the animal is a mammal. More preferably the animal is a human.

Preferred features of the fifth aspect are as defined in relation to the first, second, third and fourth aspects.

The invention will now be further described with reference to the following non-limiting examples.

### EXAMPLE 1

2-Amino-5-dialkylaminophenylthiosulphonic acids were prepared according to the following general method:

N,N-Dialkyl-p-phenylenediamine sulphate (130 mmol) was added to a mechanically stirred solution of aluminium sulfate octadecahydrate/water (43.6 g, 65 mmol/100 ml). To this was added sodium thiosulfate/water (22.0 g, 139 mmol/80 ml) followed by zinc chloride/water (8.8 g, 63 mmol/12 ml). The solution was cooled to 0° C. and potassium dichromate/water (5.0 g, 17 mmol/20 ml) was added dropwise over a 30 minute period. Following this addition, the mixture was allowed to stir for 2 hours. During the last 30 minutes the temperature was allowed to rise to 10° C. causing the formation of a viscous precipitate. This was isolated by filtration and washed with water followed by acetone.

### EXAMPLE 1A

2-Amino-5-dimethylaminophenylthiosulphonic acid was prepared as above from N,N-dimethyl-p-phenylenediamine sulphate, yield=15.87 g (49%), m.p. 190° C. (dec.)

### EXAMPLE 1B

2-Amino-5-diethylaminophenylthiosulphonic acid was prepared as above from N,N-diethyl-p-phenylenediamine sulphate, yield=17.32 g (49%), m.p. 196° C. (dec.)

### EXAMPLES 2 AND 3

Compounds of formula (III) were prepared according to the following general procedure:

The requisite thiosulphonic acid (4 mmol) and 2-substituted aniline (5 mmol) were refluxed in 120 ml methanol and silver carbonate on celite (5 g, 50% w/w) was added slowly over 0.5 h. The reaction mixture was refluxed for a further hour, filtered through a celite pad and the filtrates evaporated. The residue was extracted with dichloromethane and purified by column chromatography on silica.

### EXAMPLE 2A

### Amino-7-(dimethylamino)-2-ethylphenothiazinium hydrogensulphate

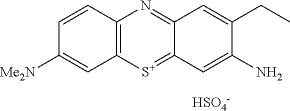

From 2-amino-5-dimethylaminobenzenethiosulphonic acid and 2-ethylaniline as violet-blue crystals, yield=273 mg, 18%; m/z, C₁₆H₁₈N₃S requires 284.40, found 284.42; λₘₐₓ (MeOH) 634 nm.

### EXAMPLE 2B

### 3-Amino-7-(dimethylamino)-2-n-propylphenothiazinium hydrogensulphate

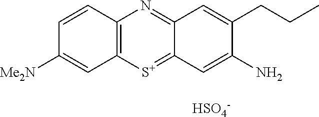

From 2-amino-5-dimethylaminobenzenethiosulphonic acid and 2-n-propylaniline as blue-black powder, yield=302 mg, 19%; m/z, C₁₇H₂₀N₃S requires 298.43, found 298.40; λₘₐₓ (MeOH) 636 nm.

### EXAMPLE 2C

### 3-Amino-2-tert-butyl-7-(dimethylamino)phenothiazinium hydrogensulphate

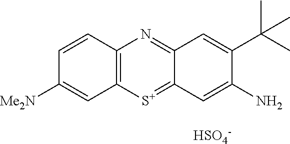

From 2-amino-5-dimethylaminobenzenethiosulphonic acid and 2-tert-butylaniline as black powder, yield=487 mg, 30%; m/z, C₁₈H₂₂N₃S requires 312.45, found 312.41; λₘₐₓ (MeOH) 637 nm.

### EXAMPLE 2D

### 3-Amino-7-(dimethylamino)-2-phenylphenothiazinium hydrogensulphate

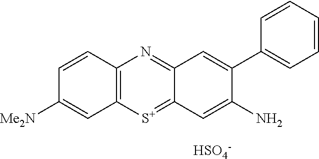

From 2-amino-5-dimethylaminobenzenethiosulphonic acid and 2-aminobiphenyl as dark blue powder, yield=455 mg, 26%; m/z, C₂₀H₁₈N₃S requires 332.44, found 332.41; λₘₐₓ (MeOH) 639 nm.

### EXAMPLE 2E

### 3-Amino-2-benzyl-7-(dimethylamino)phenothiazinium hydrogensulphate

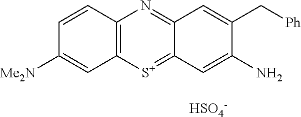

From 2-amino-5-dimethylaminobenzenethiosulphonic acid and 2-benzylaniline as blue-black powder, yield=446 mg, 25%; m/z, C₂₁H₂₀N₃S requires 346.47, found 346.47; λₘₐₓ (MeOH) 634 nm.

### EXAMPLE 2F

### 3-Amino-7-(dimethylamino)-2-methoxyphenothiazinium hydrogensulphate

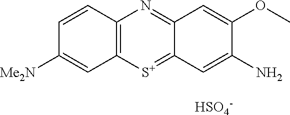

From 2-amino-5-dimethylaminobenzenethiosulphonic acid and 2-methoxyaniline as purple-blue powder, yield=384 mg, 25%; m/z, C₁₅H₁₆N₃OS requires 286.37, found 286.32; λₘₐₓ (MeOH) 639 nm.

### EXAMPLE 2G

### 3-Amino-7-(dimethylamino)-2-ethoxyphenothiazinium hydrogensulphate

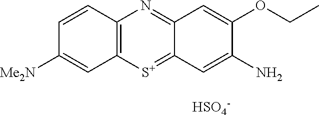

From 2-amino-5-dimethylaminobenzenethiosulphonic acid and 2-ethoxyaniline as purple-blue powder, yield=413 mg, 26%; m/z, C₁₆H₁₈N₃OS requires 300.40, found 300.38; λₘₐₓ (MeOH) 643 nm.

### EXAMPLE 3A

### Amino-7-(diethylamino)-2-ethylphenothiazinium hydrogensulphate

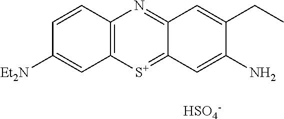

From 2-amino-5-diethylaminobenzenethiosulphonic acid and 2-ethylaniline as violet-blue crystals, yield=311 mg, 19%; m/z, C₁₈H₂₂N₃S requires 312.15, found 312.15; λₘₐₓ (MeOH) 636 nm.

### EXAMPLE 3B

### 3-Amino-7-(diethylamino)-2-n-propylphenothiazinium hydrogensulphate

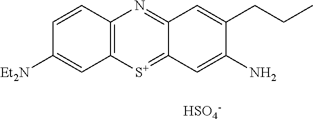

From 2-amino-5-diethylaminobenzenethiosulphonic acid and 2-n-propylaniline as blue-black powder, yield=421 mg, 25%; m/z, C₁₉H₂₄N₃S requires 326.17, found 326.06; λₘₐₓ (MeOH) 637 nm.

### EXAMPLE 3C

### 3-Amino-2-tert-butyl-7-(diethylamino)phenothiazinium hydrogensulphate

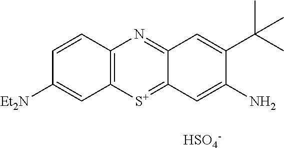

From 2-amino-5-diethylaminobenzenethiosulphonic acid and 2-tert-butylaniline as black powder, yield=418 mg, 24%; m/z, C₂₀H₂₆N₃S requires 340.18, found 340.16; λₘₐₓ (MeOH) 639 nm.

### EXAMPLE 3D

### 3-Amino-7-(diethylamino)-2-phenylphenothiazinium hydrogensulphate

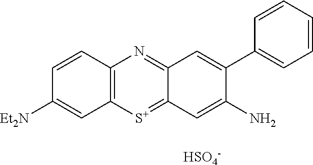

From 2-amino-5-diethylaminobenzenethiosulphonic acid and 2-aminobiphenyl as dark blue powder, yield=478 mg, 26%; m/z, C₂₂H₃₃N₃S requires 360.49, found 360.48; λₘₐₓ (MeOH) 646 nm.

### EXAMPLE 3E

### 3-Amino-2-benzyl-7-(diethylamino)phenothiazinium hydrogensulphate

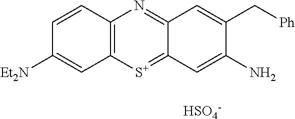

From 2-amino-5-diethylaminobenzenethiosulphonic acid and 2-benzylaniline as blue-black powder, yield=502 mg, 27%; m/z, C₂₃H₂₄N₃S requires 374.52, found 374.52; λₘₐₓ (MeOH) 642 nm.

### EXAMPLE 3F

### 3-Amino-7-(diethylamino)-2-methoxyphenothiazinium hydrogensulphate

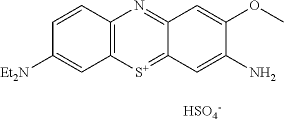

From 2-amino-5-diethylaminobenzenethiosulphonic acid and 2-methoxyaniline as purple-blue powder, yield=403 mg, 24%; m/z, C₁₇H₂₀N₃OS requires 314.42, found 314.32; λₘₐₓ (MeOH) 649 nm.

### EXAMPLE 3G

### 3-Amino-7-(diethylamino)-2-ethoxyphenothiazinium hydrogensulphate

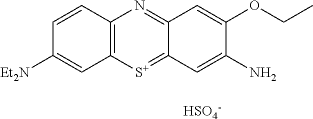

From 2-amino-5-diethylaminobenzenethiosulphonic acid and 2-ethoxyaniline as purple-blue powder, yield=482 mg, 28%; m/z, C₁₈H₂₂N₃OS requires 328.45, found 328.41; λₘₐₓ (MeOH) 648 nm.

### EXAMPLE 4

The photobactericidal efficacies of the compounds of examples 2a to 2d in addition to that of the known photosensitiser methylene blue were measured against both Gram positive Staphylococcus aureus (NCTC 6571), Enterococcus faecalis (NCIMB 13280) and Propionibacterium acnes (NCTC 737) and Gram negative Escherichia coli (NCTC 10418), Proteus mirabilis (NCIMB 5887) and a clinical strain of Pseudomonas aeruginosa bacteria. Propionibacterium acnes was grown in reinforced clostridial medium. All other strains were grown in Mueller-Hinton Broth and then diluted to a concentration of 10⁶ colony-forming units/ml. Aliquots of the strains were then incubated for 1 hour at 37° C. in microtitre trays with various concentrations of photosensitiser in doubling dilutions from 100 μM, with zero photosensitiser concentrations in each case for control purposes. The trays were then either illuminated for twenty minutes using an array of light-emitting diodes (660 nm) giving a light dose of 6.2 J cm⁻² or alternatively foil-covered to provide dark controls. From each well showing an inhibition of growth of the micro-organism, 1 μl was sub-cultured on nutrient agar, using the Miles-Misra method, and incubated for 18 hours at 37° C. The minimum bactericidal concentrations were then determined as the lowest concentration for each photosensitiser giving no bacterial growth.

Antifungal screening was carried out similarly against the yeast Candida albicans (NCPF 8179), using Sabouraud broth and agar.

Table 1 shows the minimum concentration in micromoles/litre needed to achieve the antibacterial/antifungal activity. As can be seen, the compounds of the present invention are more active toluidine blue.

TABLE 1

| | S. | Enterococcus | Prop. | E. | Proteus | Pseudomonas | Candida |
|---|---|---|---|---|---|---|---|
| Compound | aureus | facecalis | acnes | coli | mirabilis | Aeruginosa | albicans |
| toluidine | 7.36 | 7.36 | 6.25 | 7.36 | 14.71 | 7.36 | 12.5 |
| blue | | | | | | | |
| 2a | 3.13 | 1.35 | 0.78 | 1.47 | 5.50 | 3.13 | 3.13 |
| 2b | 3.13 | 0.33 | 0.78 | 1.32 | 5.32 | 0.78 | 0.78 |
| 2c | 3.13 | | 0.78 | 3.13 | | 0.39 | 3.13 |
| 2d | 3.13 | | | 3.13 | | | 0.78 |

1. (canceled)

2. The compound of claim 12, wherein R¹ is selected from ethyl, propyl (especially n-propyl), butyl (especially t-butyl), phenyl, benzyl, methoxy and ethoxy.

3. The compound of claim 12, wherein each of R³ and R⁴ is independently selected from methyl, ethyl, n-propyl, n-butyl, t-butyl, n-pentyl and n-hexyl.

4. The compound of claim 12, wherein R² is hydrogen.

5. The compound of claim 12, wherein the compound has the structure shown in formula (IV): 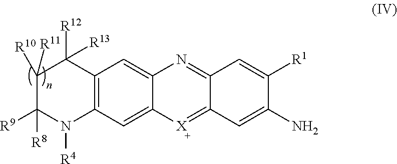
wherein n is suitably 0 to 2, preferably 1, R⁴ is preferably alkyl, and each of R⁸, R⁹, R¹⁰, R¹¹, R¹² and R¹³ is independently hydrogen or an optionally substituted alkyl, alkenyl, alkoxy or aryl group.

6. The compound of claim 12, wherein X is sulphur.

7. The compound of claim 12, wherein X is oxygen.

8. The compound of claim 15, wherein the pathogen is selected from a group consisting of; viruses, bacteria, fungi, and protozoa.

9. (canceled)

10. The compound of claim 15, wherein the tumour cells are located at or near to a surface of the body.

11. A composition comprising the compound of claim 12 and a pharmaceutically acceptable carrier.

12. A compound of formula (III): 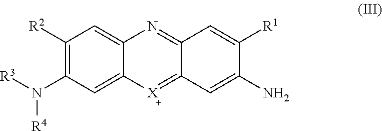
or a pharmaceutically acceptable derivative thereof;
wherein X is selected from O, S and Se; each of R², R³ and R⁴ is independently selected from hydrogen or an optionally substituted alkyl, alkenyl, alkynyl or aryl group; and R¹ is selected from halogen, sulfo, acyl, sulfoxy, mercapto, nitro, amino, hydroxy or an optionally substituted alkyl, alkenyl, alkynyl, aryl, amine or alkoxy group; wherein R¹ is not methyl or hydrogen when each of R³ and R⁴ is methyl or hydrogen.

13. (canceled)

14. A method of treating or diagnosing an animal having a disease caused by a pathogen or tumour cells, the method comprising administering to the animal a compound 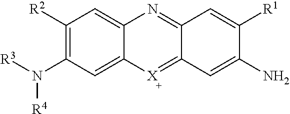
according to claim 12.

15. A method of combatting and/or detecting a pathogen and/or tumour cells, the method comprising:
exposing, in the presence of oxygen, a compound according to formula III to light having a wavelength between about 500 nm and about 900 nm 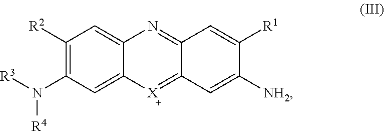
wherein X is selected from O, S and Se; each of R², R³ and R⁴ is independently selected from hydrogen or an optionally substituted alkyl, alkenyl, alkynyl or aryl group; and R¹ is selected from halogen, sulfa, acyl, sulfoxy, mercapto, nitro, amino, hydroxy or an optionally substituted alkyl, alkenyl, alkynyl, aryl, amine or alkoxy group; wherein R¹ is not methyl or hydrogen when each of R³ and R⁴ is methyl or hydrogen.

16. A method of preparing a compound of formula (III) 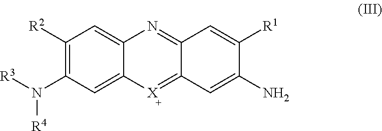
or a pharmaceutically acceptable derivative thereof, wherein X is selected from O, S and Se; each of R², R³ and R⁴ is independently selected from hydrogen or an optionally substituted alkyl, alkenyl, alkynyl or aryl group; and R¹ is selected from halogen, sulfo, acyl, sulfoxy, mercapto, nitro, amino, hydroxy or an optionally substituted alkyl, alkenyl, alkynyl, aryl, amine or alkoxy group; wherein R¹ is not methyl or hydrogen when each of R³ and R⁴ is methyl or hydrogen, the method comprising:
in the case that X is S, reacting a compound of formula (VI) with a compound of formula (VII) under oxidative conditions, according to Equation I: 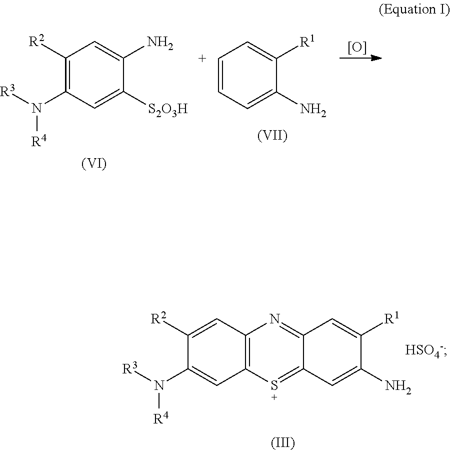
in the case that X is O, reacting a compound of formula (VIII) with a compound of formula (IX) under oxidative conditions, according to Equation II: 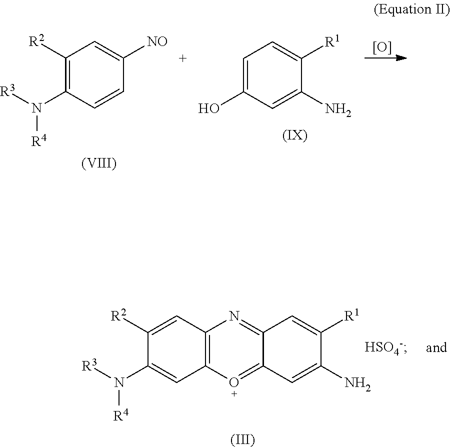
in the case that X is Se, reacting a compound of formula (X) with a compound of formula (XI) under oxidative conditions, according to Equation (III) [chemical structure]
